(19) United States
(12) Patent Application Publication
(10) Pub. No.: US 2006/0167048 A1
(43) Pub. Date: Jul. 27, 2006

(54) N-4-piperidinyl compounds as ccr5 modulators

(76) Inventors: John Cumming, Macclesfield (GB); Jon Winter, Macclesfield (GB)

Correspondence Address: FISH & RICHARDSON P.C., P.O BOX 1022, MINNEAPOLIS, MN, 55440-1022, US

(21) Appl. No.: 10/525,121

(22) PCT Filed: Aug. 19, 2003
(86) PCT No.: PCT/SE03/01287

§ 371 Date: Feb. 18, 2005

(30) Foreign Application Priority Data
Aug. 21, 2002 (SE) 0202483-4

(51) Int. Cl. C07D 401/02; A61K 31/4545

(52) U.S. Cl. 514/316; 546/189

(57) ABSTRACT

The invention provides a compound of formula (I): [Chemical formula should be inserted here. Please see paper copy.] wherein R₁, R₂, R₃, R₃ₐ, R₄, R₄ₐ, R₅, and R₆ are as defined; or a pharmaceutically acceptable salt thereof or a solvate thereof; compositions containing these compounds, processes for preparing them and their use as modulators of chemokine activity (especially CCR5 activity).

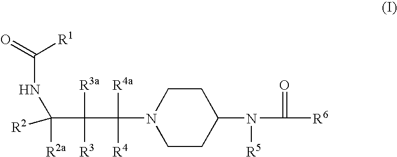

The present invention relates to heterocyclic derivatives having pharmaceutical activity, to processes for preparing such derivatives, to pharmaceutical compositions comprising such derivatives and to the use of such derivatives as active therapeutic agents.

Pharmaceutically active piperidine derivatives are disclosed in PCT/SEQ01/01053, EP-A1-1013276, WO00/08013, WO99/38514 and WO99/04794.

Chemokines are chemotactic cytokines that are released by a wide variety of cells to attract macrophages, T cells, eosinophils, basophils and neutrophils to sites of inflammation and also play a role in the maturation of cells of the immune system. Chemokines play an important role in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved four cysteine motif. The chemokine superfamily can be divided into two main groups exhibiting characteristic structural motifs, the Cys-X-Cys (C-X-C, or α) and Cys-Cys (C-C, or β) families. These are distinguished on the basis of a single amino acid insertion between the NH-proximal pair of cysteine residues and sequence similarity.

The C-X-C chemokines include several potent chemoattractants and activators of neutrophils such as interleukin-8 (IL-8) and neutrophil-activating peptide 2 (NAP-2).

The C-C chemokines include potent chemoattractants of monocytes and lymphocytes but not neutrophils such as human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β).

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10, CXCR1, CXCR2, CXCR3 and CXCR4. These receptors represent good targets for drug development since agents which modulate these receptors would be useful in the treatment of disorders and diseases such as those mentioned above.

The CCR5 receptor is expressed on T-lymphocytes, monocytes, macrophages, dendritic cells, microglia and other cell types. These detect and respond to several chemokines, principally “regulated on activation normal T-cell expressed and secreted” (RANTES), macrophage inflammatory proteins (MIP) MW-1a and MP-1b and monocyte chemoattractant protein-2 (MCP-2).

This results in the recruitment of cells of the immune system to sites of disease. In many diseases it is the cells expressing CCR5 which contribute, directly or indirectly, to tissue damage. Consequently, inhibiting the recruitment of these cells is beneficial in a wide range of diseases.

CCR5 is also a co-receptor for HIV-1 and other viruses, allowing these viruses to enter cells. Blocking the receptor with a CCR5 antagonist or inducing receptor internalisation with a CCR5 agonist protects cells from viral infection.

The present invention provides a compound of formula (I): 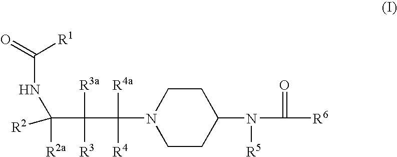
wherein:
R¹ is C₁₋₆ alkoxy (optionally substituted by C₁₋₄ alkoxy or phenyl), C₃₋₆ alkenyloxy, phenoxy or piperidin-4-yl (1-substituted by C(O)R⁷ or S(O)₂R⁸);
R² is optionally substituted phenyl, optionally substituted heteroaryl or cycloalkyl;
R^{2a}, R⁴ and R^{4a} are, independently, hydrogen or C₁₋₄ alkyl;
R³ and R^{3a} are, independently, hydrogen or C₁₋₄ alkyl or C₁₋₄ alkoxy;
R⁵ is hydrogen, C₁₋₄ alkyl (optionally substituted by halogen, hydroxy, C₁₋₄ alkoxy, C₃₋₇ cycloalkyl, SH, C₁₋₄ alkylthio, cyano or S(O)_{q}(C₁₋₄ alkyl)), C₃₋₄ alkenyl, C₃₋₄ alkynyl or C₃₋₇ cycloalkyl;
R⁶ is phenyl, heteroaryl, phenylNH, heteroarylNH, phenyl(C₁₋₂)alkyl, heteroaryl(C₁₋₂)alkyl, phenyl(C₁₋₂ alkyl)NH or heteroaryl(C₁₋₂ alkyl)NH;
R⁷ is C₁₋₆ alkyl (optionally substituted by phenyl, heteroaryl, C₁₋₄ alkoxy, or C₁₋₄ alkoxy(C₁₋₄ alkoxy)), C₁₋₆ alkoxy, phenyl, heteroaryl or C₃₋₆ cycloalkyl;
R⁸ is C₁₋₆ alkyl (optionally substituted by phenyl) or phenyl; wherein the phenyl and heteroaryl rings of any of the foregoing are independently optionally substituted by halo, cyano, nitro, hydroxy, C₁₋₄ alkyl, C₁₋₄ alkoxy, S(O)ₘC₁₋₄ alkyl, S(O)₂NR⁹R¹⁰, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃;
R⁹ and R¹⁰ are, independently, hydrogen or C₁₋₄ alkyl, or together with a nitrogen or oxygen atom, may join to form a 5- or 6-membered ring which is optionally substituted with C₁₋₄ alkyl, C(O)H or C(O)(C₁₋₄ alkyl);
m, p and q are, independently, 0, 1 or 2;
or a pharmaceutically acceptable salt thereof or a solvate thereof.

Certain compounds of the present invention can exist in different isomeric forms (such as enantiomers, diastereomers, geometric isomers or tautomers). The present invention covers all such isomers and mixtures thereof in all proportions.

Suitable salts include acid addition salts such as a hydrochloride, hydrobromide, phosphate, acetate, fumarate, maleate, tartrate, citrate, oxalate, methanesulphonate or p-toluenesulphonate.

The compounds of the invention may exist as solvates (such as hydrates) and the present invention covers all such solvates.

Alkyl groups and moieties preferably contain, unless otherwise specified, 1-6, especially 1-4, carbon atoms. Alkyl groups and moieties are straight or branched chain and are, for example, methyl, ethyl, n-propyl, iso-propyl or tert-butyl.

Alkenyl and alkynyl groups and moieties preferably contain, unless otherwise specified, 2-6, especially 24, carbon atoms. Alkenyl includes prop-2-en-1-yl, allyl, but-3-en-1-yl, but-1-en-1-yl, 2-methylallyl, 1-methyl-but-3-en-1-yl, 1-methyl-but-1-en-1-yl, pent-2-en-1-yl and hex-1-en-1-yl. Alkynyl includes propargyl, but-3-yn-1-yl, pent-4-yn-1-yl and hex-5-yn-1-yl. Alkenyl and alkynyl groups and moieties are, for example, vinyl, allyl or propargyl.

Cycloalkyl preferably contains, unless otherwise specified, 3-7, especially 36, carbon atoms. Cycloalkyl is, for example, cyclopropyl, cyclobutyl or cyclopentyl.

Cycloalkyl fused to a phenyl ring is, for example, benzocyclobuten-1-yl, indan-1-yl or indan-2-yl.

Heteroaryl is an aromatic 5 or 6 membered ring comprising at least one heteroatom selected from the group comprising nitrogen, oxygen and sulphur. Heteroaryl is, for example, pyrrolyl, imidazolyl, pyrazolyl, 1,2,3-triazolyl, 1,2,4-triazolyl, oxazolyl, isoxazolyl, oxadiazolyl, thiazolyl, isothiazolyl, thiadiazolyl, pyridinyl, pyrimidinyl, pyrazinyl, pyridazinyl, thienyl, furyl, quinolinyl, isoquinolinyl, dihydroisoquinolinyl, quinazolinyl, quinoxalinyl, indolyl, isoindolyl, benzimidazolyl, benzo[b]furyl, benzo[b]thienyl, phthalazinyl, benzthiazolyl or cinnolinyl.

Phenylalkyl is, for example, benzyl, 1-(phenyl)eth-1-yl or 1-(phenyl)eth-2-yl.

Heteroarylalkyl is, for example, pyridinylmethyl, pyrimidinylmethyl or 1-(pyridinyl)eth-2-yl.

The group S(O)₂NR⁹R¹⁰ is, for example, S(O)₂NH₂, S(O)₂NH(C₁₋₄ alkyl), S(O)₂N(C₁₋₄ alkyl)₂, S(O)₂(4-C(O)H-piperazin-1-yl) or S(O)₂(4-C(O)CH₃-piperazin-1-yl).

Phenyl(C₁₋₂ alkyl)NH is, for example, benzylamino. Heteroaryl(C₁₋₂ alkyl)NH is, for example, pyridinylCH₂NH, pyrimidinylCH₂NH or pyridinylCH(CH₃)NH.

In one aspect the present invention provides a compound of formula (I), wherein R¹ is piperidinyl (1-substituted by C(O)R⁷ or S(O)₂R⁸), wherein R⁷ and R⁸ are as defined above.

In a further aspect R¹ is piperidinyl-4-yl 1-substituted by C(O)R⁷, wherein R⁷ is as defined above. R⁷ is, for example, C₁₋₆ alkyl (optionally mono-substituted by phenyl), C₁₋₆ alkoxy, phenyl or C₃₋₆ cycloalkyl, wherein the phenyl rings are optionally substituted by halogen.

In a still further aspect R¹ is piperidin-4-yl 1-substituted by S(O)₂R⁸, wherein R⁸ is as defined above. R⁸ is, for example, phenyl or C₁₋₆ alkyl (optionally mono-substituted by phenyl), wherein the phenyl rings are optionally substituted by halogen, S(O)₂(C₁₋₄ alkyl) or NHC(O)(C₁₋₄ alkyl).

In another aspect R¹ is C₁₋₆ alkoxy (optionally substituted by C₁₋₄ alkoxy or phenyl), C₃₋₆ alkenyloxy or phenoxy (optionally substituted by halogen).

In yet another aspect of the present invention R¹ is piperidin-4-yl 1-substituted by C(O)R⁷ (wherein R⁷ is C₁₋₆ alkyl (optionally mono-substituted by phenyl), C₁₋₆ alkoxy, phenyl or C₃₋₆ cycloalkyl, wherein the phenyl rings are optionally substituted by halogen) or S(O)₂R⁸ {wherein R⁸ is phenyl or C₁₋₆ alkyl (optionally mono-substituted by phenyl), wherein the phenyl rings are optionally substituted by halogen, S(O)₂(C₁₋₄ alkyl) or NHC(O)(C₁₋₄ alkyl)}, or R¹ is C₁₋₆ alkoxy (optionally substituted by C₁₋₄ alkoxy or phenyl), C₃₋₆ alkenyloxy or phenoxy (optionally substituted by halogen).

In a further aspect R² is phenyl optionally substituted (such as in the ortho or meta position) by halo, C₁₋₄ alkyl, C₁₋₄ alkoxy, S(O)ₙ(C₁₋₄ alkyl) (wherein n is 0, 1 or 2), nitro, cyano or CF₃. Halo is especially fluorine or chlorine. For example R² is unsubstituted phenyl, 3-fluorophenyl, 3-chlorophenyl, 4-fluorophenyl or 4-CF₃-phenyl. R² can additionally be 3,5-difluorophenyl.

In a still further aspect R² is phenyl optionally substituted (such as in one of both meta positions) by halo (for example fluorine), C₁₋₄ alkyl, cyano or CF₃. For example R² is unsubstituted phenyl, 3-fluorophenyl or 3,5-difluorophenyl.

In another aspect R² is phenyl optionally substituted in the ortho or meta position by fluorine or chlorine. For example R² is unsubstituted phenyl, 3-fluorophenyl or 3-chlorophenyl.

In yet anther aspect R² is phenyl optionally substituted (such as in one of both meta positions) by halo (for example fluorine). For example R² is unsubstituted phenyl, 3-fluorophenyl or 3,5-difluorophenyl.

In another aspect R^{2a} is hydrogen.

In yet another aspect R³ and R^{3a} are both hydrogen.

In a yet further aspect R⁴ and R^{4a} are, independently, hydrogen or methyl (for example R⁴ is hydrogen and R^{4a} is methyl, or, R⁴ and R^{4a} are both hydrogen).

In a still further aspect R⁴ is hydrogen or methyl and R^{4a} is hydrogen.

In a still further aspect R^{2a}, R³, R^{3a}, R⁴ and R^{4a} are all hydrogen.

In another aspect R⁵ is hydrogen, C₁₋₄ alkyl (such as methyl, ethyl or iso-propyl), C₃₋₄ alkenyl, C₃₋₄ alkynyl, C₃₋₇ cycloalkyl or C₃₋₇ cycloalkyl(C₁₋₄ alkyl). For example R⁵ is hydrogen, methyl, ethyl, allyl, propargyl, cyclopropyl or cyclopropylCH₂.

In a further aspect R⁵ is hydrogen, methyl, ethyl, allyl or cyclopropyl.

In still further aspects of the invention R⁵ is ethyl.

In another aspect R⁶ is phenyl or benzyl; the phenyl rings of these being optionally substituted by one or more of: halo, cyano, nitro, hydroxy, C₁₋₄ alkyl, C₁₋₄ alkoxy, S(O)ₘC₁₋₄ alkyl, S(O)₂NR⁹R¹⁰, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃; wherein m, R⁹ and R¹⁰ are as defined above or below.

In a still further aspect R⁶ is benzyl the phenyl ring of which is optionally substituted by one or more of: halo, cyano, nitro, hydroxy, C₁₋₄ allyl, C₁₋₄ alkoxy, S(O)ₘC₁₋₄ alkyl, S(O)₂NR⁹R¹¹, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H; CO₂(C₁₋₄ alkyl), C(O)(C₁₋₄ alkyl), CF₃, CHF₂, CH₂F, CH₂CF₃ or OCF₃; wherein m, R⁹ and R¹⁰ are as defined above or below.

In another aspect of the invention R⁹ and R¹⁰ are, independently, hydrogen or C₁₋₄ alkyl.

In yet another aspect of the invention R⁶ is benzyl singly substituted (such as in the 4 position) by S(O)₂(C₁₋₄)alkyl (such as S(O)₂CH₃) or S(O)₂NR⁹R¹⁰ {R⁹ and R¹⁰ are, independently, hydrogen or C₁₋₄ alkyl, or together with a nitrogen or oxygen atom, may join to form a 5- or 6-membered ring which is optionally substituted with C₁₋₄ alkyl, C(O)H or C(O)(C₁₋₄ alkyl)} (such as S(O)₂NH₂, S(O)₂NH(CH₃), S(O)₂N(CH₃)₂, S(O)₂(4-C(O)H-piperazin-1-yl) or S(O)₂(4-C(O)CH₃-piperazin-1-yl). The 5- or 6-membered ring is, for example, morpholine, thiomorpholine, piperidine, piperazine or pyrrolidine; but is especially piperazine.

In another aspect of the invention R⁶ is benzyl singly substituted (such as in the 4-position) by S(O)₂(C₁₋₄)alkyl (such as S(O)₂CH₃).

In a further aspect the present invention provides a compound of formula (I) wherein R¹ is piperidin-4-yl 1-substituted by C(O)R⁷ {wherein R⁷ is C₁₋₆ alkyl (optionally mono-substituted by phenyl), C₁₋₆ alkoxy, phenyl or C₃₋₆ cycloalkyl, wherein the phenyl rings are optionally substituted by halogen} or S(O)₂R⁸ {wherein R^{a} is phenyl or C₁₋₆ alkyl (optionally mono-substituted by phenyl), wherein the phenyl rings are optionally substituted by halogen, S(O)₂(C₁₋₄ alkyl) or NHC(O)(C₁₋₄ alkyl)}, or R¹ is C₁₋₆ alkoxy {optionally substituted by C₁₋₄ alkoxy or phenyl), C₃₋₆ alkenyloxy or phenoxy (optionally substituted by halogen}; R² is phenyl or phenyl substituted in one or both meta-positions by halogen (for example 3-fluorophenyl); R², R³, R^{3a}, R⁴ and R^{4a} are all hydrogen; R⁵ is ethyl; and, R⁶ benzyl wherein the phenyl ring is substituted by S(O)₂(C₁₋₄ alkyl) (for example S(O)₂CH₃); or a pharmaceutically acceptable salt thereof (such as a salt of a hydro-halogen acid, for example a hydrochloride).

In yet another aspect the present invention provides a compound of formula (Ia): 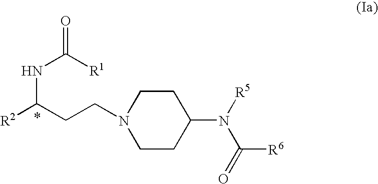
wherein R¹, R⁵ and R⁶ are as defined above, and R² is unsubstituted phenyl or 3-fluorophenyl. In a further aspect the invention provides a compound of formula (I) wherein R¹, R⁵ and R⁶ are as defined above, and R² is unsubstituted phenyl, 3-fluorophenyl or 3,5-difluorophenyl. In another aspect of the invention the compounds of formula (Ia) have S absolute configuration at the asterisked carbon (that is, the carbon labelled ‘*’).

In a further aspect the present invention provides a compound of formula (Ib): 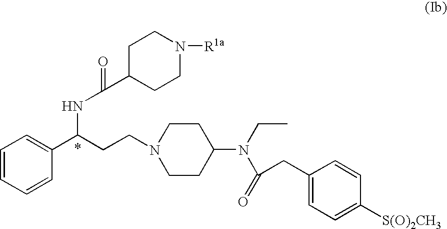
having S absolute configuration at *, wherein R^{1a} is piperidin-4-yl 1-substituted by: C(O)R⁷ or S(O)₂R⁸, wherein R⁷ is as defined above {for example R⁷ is C₁₋₆ alkyl (optionally mono substituted by phenyl), C₁₋₆ alkoxy, phenyl or C₃₋₆ cycloalkyl, wherein the phenyl rings are optionally substituted by halogen} and R⁸ is as defined above {for example R⁸ is phenyl or C₁₋₆ alkyl (optionally mono-substituted by phenyl), wherein the phenyl rings are optionally substituted by halogen, S(O)₂(C₁₋₄ alkyl) or NHC(O)(C₁₋₄ alkyl)}; or a pharmaceutically acceptable salt thereof (such as a salt of a hydro-halogen acid, for example a hydrochloride).

In a still further aspect the present invention provides a compound of formula (Ic): 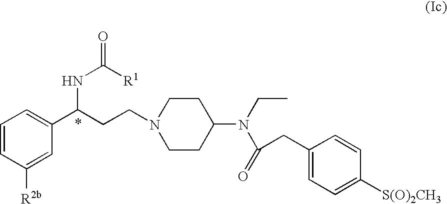
having S absolute configuration at *, wherein R¹ is as defined-above (for example R¹ is C₁₋₆ alkoxy (optionally substituted by C₁₋₄ alkoxy or phenyl), C₃₋₆ alkenyloxy or phenoxy (optionally substituted by halogen); and R^{2b} is hydrogen or halogen (for example fluoro).

The following compounds illustrate the invention.

TABLE I
Table I comprises compounds of formula (Ib), all having S absolute configuration at *.

| | |
|---|---|
| | (Ib) |
| 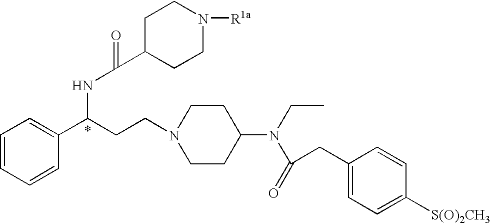 | |

| Compound | | LCMS |
|---|---|---|
| No. | R^{1a} | (MH+) |
| 1 | C(O)CH₃ | 611 |
| 2 | C(O)OC(CH₃)₃ | 669 |
| 3 | S(O)₂Phenyl | 709 |
| 4 | S(O)₂CH(CH₃)₂ | 675 |
| 5 | S(O)₂CH₃ | 647 |
| 6 | S(O)₂CH₂Phenyl | 723 |
| 7 | S(O)₂CH₂CH₃ | 661 |
| 8 | S(O)₂(4-S(O)₂CH₃—C₆H₄) | 787 |
| 9 | S(O)₂(4-F—C₆H₄) | 727 |
| 10 | S(O)₂(4-NHC(O)CH₃—C₆H₄) | 766 |
| 11 | C(O)CH(CH₃)₂ | 639 |
| 12 | C(O)CH₂Phenyl | 687 |
| 13 | C(O)CH₂O(CH₂)₂OCH₃ | 685 |
| 14 | C(O)Cyclopropyl | 637 |
| 15 | C(O)(4-Cl-C₆H₄) | 751 |
| 16 | C(O)Phenyl | 673 |

TABLE II
Table II comprises compounds of formula (Ic), all having S absolute configuration at
*.

| | |
|---|---|
| | (Ic) |
| 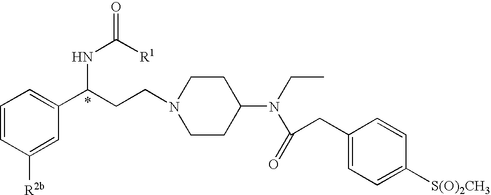 | |

| Compound No. | R¹ | R^{2b} | Salt | LCMS (MH+) |
|---|---|---|---|---|
| 1 | OC(CH₃)₃ | H | | 558 |
| 2 | OCH₂CH(CH₃)₂ | H | HCl | 558 |
| 3 | O(CH₂)₂OCH₃ | H | HCl | 560 |
| 4 | Phenoxy | H | HCl | 578 |
| 5 | Benzyloxy | H | HCl | 592 |
| 6 | O(4-F—C₆H₄) | H | HCI | 596 |
| 7 | OCH₂CCl₃ | H | HCl | 634 |
| 8 | OCH₂CH═CH₂ | H | HCl | 542 |
| 9 | O(4-Cl—C₆H₄) | H | HCl | 612 |
| 10 | OC(CH₃)₃ | F | | 576 |

A compound of formula (I), (Ia), (Ib) or (Ic) can be prepared by treating a compound formula (II): 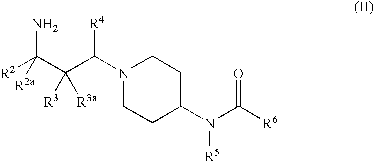
with:
an acid chloride or chloroformate of formula R¹C(O)Cl, in the presence of a base (such as a tertiary amine, such as N(C₁₋₆ alkyl)₃ where the alkyl groups can be the same, two of the same or all different, for example triethylamine) and in a suitable solvent (such as a chlorinated hydrocarbon, for example dichloromethane); or
when R¹ is a 1-substituted piperidinyl, an acid of formula R¹CO₂H in the presence of a suitable coupling agent (such as O-(7-azabenzotriazol-1-yl)-N,N,N′,N′-tetramethyluronium hexafluorophosphate [HATU] or bromo-tris-pyrrolidino-phosphonium hexafluorophosphate [PyBrop]) in the presence of a suitable base (such as a tertiary amine, for example diisopropylethylamine) in a suitable solvent (such as N-methylpyrrolidinone).

A compound of formula (II) can be prepared by treating a compound of formula (III): 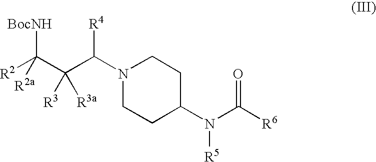
with trifluoroacetic acid or hydrochloric acid in the presence of methanol, and then basifying to release the free amine form of formula (II).

A compound of formula (III) can be prepared by reductively aminating a compound of formula (IV): 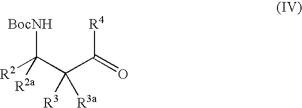
with a compound of formula (V): 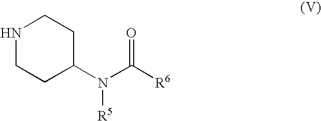
in the presence of a suitable solvent (such as an aliphatic alcohol such as methanol), a suitable organic acid (such as an aliphatic acid, for example acetic acid) and a suitable reducing agent (such as sodium triacetoxyborohydride or sodium cyanoborohydride).

A compound of formula (II) wherein R^{2a} is hydrogen can be prepared by reductive amination of a compound of formula (VI): 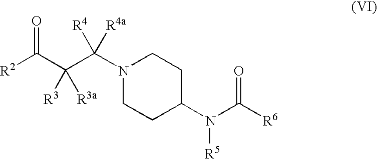
for example by reacting a compound of formula (VI) with hydroxylamine and hydrogenating the product so formed with hydrogen in the presence of a suitable metal catalyst (such as palladium or platinum catalyst, for example palladium on charcoal).

A compound of formula (VI), wherein R^{4a} is hydrogen, can be prepared by reacting a compound of formula (V) with:

an alkyl halide of formula R²C(O)CR³R^{3a}CHR⁴X (wherein X is halogen, such as chloro, bromo or iodo) in the presence of a suitable base (such as potassium carbonate) and a suitable solvent (such as acetone); or,

compounds of formula R²C(O)CHR³R^{3a} and R⁴CHO in the presence of a suitable acid (such as acetic acid.

A compound of formula (VI), wherein R^{3a} is hydrogen, can be prepared by reacting a compound of formula (V) with an alkene of formula R²C(O)CR³═CR⁴R^{4a} in a suitable solvent (such as an aliphatic alcohol, for example ethanol) at a temperature in the range −10 to 100° C.

Alternatively, a compound of formula (I) can be prepared by reacting a compound of formula (VII): 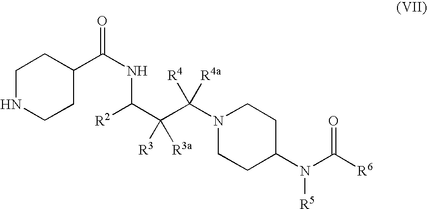
with an acid chloride R⁷C(O)Cl or sulfonyl chloride R⁸S(O)₂Cl in the presence of a base (such as a tertiary amine, such as N(C₁₋₆ alkyl)₃ where the alkyl groups can be the same, two of the same or all different, for example triethylamine) and in a suitable solvent (such as a chlorinated hydrocarbon, for example dichloromethane); or
with an acid of formula R⁷CO₂H in the presence of a suitable coupling agent (such as O-(7-azabenzotriazol-1-yl)-N,N,N′,N′-tetramethyluronium hexafluorophosphate [HATU] or bromo-tris-pyrrolidino-phosphonium hexafluorophosphate [PyBrop]) in the presence of a suitable base (such as a tertiary amine, for example diisopropylethylamine) in a suitable solvent (such as N-methylpyrrolidinone).

A compound of formula (VII) can be prepared by deprotecting a compound of formula (VIII): 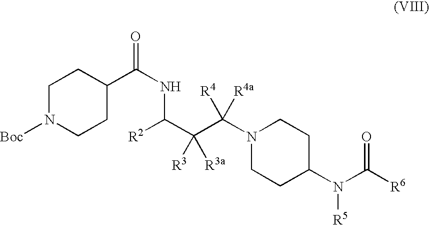
with a suitable acid (such as hydrochloric acid or trifluoracetic acid).

A compound of formula (VIII) can be prepared by reacting a compound of formula (IX): 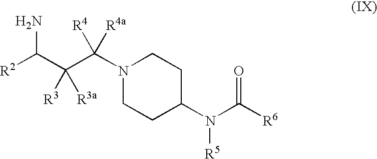
with 1-Boc-piperidine-4-carboxylic acid and a suitable coupling reagent (such as O-(7-azabenzotriazol-1-yl)-N,N,N′,N′-tetramethyluronium hexafluorophosphate [HATU] or bromo-tris-pyrrolidino-phosphonium hexafluorophosphate [PyBrop]).

The starting materials for these processes are commercially available, can be prepared by literature methods or can be prepared by adapting literature methods. In a further aspect the invention provides processes for preparing the compounds of formulae (I), (Ia), (Ib) and (Ic). Many of the intermediates in the processes are novel and these are provided as further features of the invention.

The compounds of the invention have activity as pharmaceuticals, in particular as modulators (such as agonists, partial agonists, inverse agonists or antagonists) of chemokine receptor (especially CCR5) activity, and may be used in the treatment of autoimmune, inflammatory, proliferative or hyperproliferative diseases, or immunologically-mediated diseases (including rejection of transplanted organs or tissues and Acquired Immunodeficiency Syndrome (AIDS)).

The compounds of the present invention are also of value in inhibiting the entry of viruses (such as human immunodeficiency virus (HIV)) into target calls and, therefore, are of value in the prevention of infection by viruses (such as HIV), the treatment of infection by viruses (such as HIV) and the prevention and/or treatment of acquired immune deficiency syndrome (AIDS).

According to a further feature of the invention there is provided a compound of the formula (I), (Ia), (Ib) or (Ic) {for example (I), (Ia) or (Ib)}, or a pharmaceutically acceptable salt thereof or a solvate thereof, for use in a method of treatment of a warm blooded animal (such as man) by therapy (including prophylaxis).

The present invention further provides a method of treating a chemokine mediated disease state (especially a CCR5 mediated disease state, such as rheumatoid arthritis) in a warm blooded animal (such as man) suffering from, or at risk of, said disease, which comprises administering to an animal in need of such treatment a therapeutically effective amount of a compound of formula (I), (Ia), (Ib) or (Ic) {for example (I), (Ia) or (Ib)}, or a pharmaceutically acceptable salt thereof or solvate thereof.

The invention also provides a compound of the formula (I), (Ia), (Ib) or (Ic) {for example (I), (a) or (Ib)}, or a pharmaceutically acceptable salt thereof or a solvate thereof, for use in therapy (including prophylaxis); for example in the treatment of a chemokine mediated disease state (especially a CCR5 mediated disease state) in a warm blooded animal, such as man, such as in the treatment of rheumatoid arthritis.

In another aspect the present invention provides the use of a compound of the formula (I), (Ia), (Ib) or (Ic) {for example (I), (Ia) or (Ib)}, or a pharmaceutically acceptable salt thereof or a solvate thereof, in the manufacture of a medicament for use in therapy (for example in modulating chemokine receptor activity (especially CCR5 receptor activity (especially in the treatment of rheumatoid arthritis)) in a warm blooded animal, such as man).

The invention further provides the use of a compound of formula (I), (Ia), (Ib) or (Ic) {for example (I), (Ia) or (Ib)}, or a pharmaceutically acceptable salt thereof, in the manufacture of a medicament for use in the treatment of one or more of the following disease states:

(1) (the respiratory tract) obstructive diseases of airways including: chronic obstructive pulmonary disease (COPD) (such as irreversible COPD); asthma {such as bronchial, allergic, intrinsic, extrinsic or dust asthma, particularly chronic or inveterate asthma (for example late asthma or airways hyper-responsiveness)}; bronchitis {such as eosinophilic bronchitis}; acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (bay fever) or vasomotor-rhinitis; sarcoidosis; farmer's lung and related diseases; nasal polyposis; fibroid lung or idiopathic interstitial pneumonia;

(2) (bone and joints) arthrides including rheumatic, infectious, autoimmune, seronegative spondyloarthropathies (such as ankylosing spondylitis, psoriatic arthritis or Reiter's disease), Behcet's disease, Sjogren's syndrome or systemic sclerosis;

(3) (skin and eyes) psoriasis, atopic dermatitis, contact dermatitis or other eczmatous dermitides, seborrhoetic dermatitis, Lichen planus, Phemphigus, bullous Phemphigus, Epidermolysis bullosa, urticaria, angiodermas, vasculitides erythemas, cutaneous eosinophilias, uveitis, Alopecia areata or vernal conjunctivitis;

(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);

(5) (Allograft rejection) acute and chronic following, for example, transplantation of kidney, heart, liver, lung, bone marrow, skin or cornea; or chronic graft versus host disease; and/or

(6) (other tissues or diseases) Alzheimer's disease, multiple sclerosis, atherosclerosis, Acquired Immunodeficiency Syndrome (ADS), Lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, sezary syndrome, idiopathic thrombocytopenia pupura or disorders of the menstrual cycle;

in a warm blooded animal, such as man.

In order to use a compound of the invention, or a pharmaceutically acceptable salt thereof or solvate thereof, for the therapeutic treatment of a warm blooded animal, such as man, in particular modulating chemokine receptor (for example CCR5 receptor) activity, said ingredient is normally formulated in accordance with standard pharmaceutical practice as a pharmaceutical composition.

Therefore in another aspect the present invention provides a pharmaceutical composition which comprises a compound of the formula (I), (Ia), (Ib) or (Ic) {for example (I), (Ia) or (Ib)}, or a pharmaceutically acceptable salt thereof or a solvate thereof (active ingredient), and a pharmaceutically acceptable adjuvant, diluent or carrier. In a further aspect the present invention provides a process for the preparation of said composition which comprises mixing active ingredient with a pharmaceutically acceptable adjuvant, diluent or carrier. Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The pharmaceutical compositions of this invention may be administered in standard manner for the disease condition that it is desired to treat, for example by topical (such as to the lung and/or airways or to the skin), oral, rectal or parenteral administration. For these purposes the compounds of this invention may be formulated by means known in the art into the form of, for example, aerosols, dry powder formulations, tablets, capsules, syrups, powders, granules, aqueous or oily solutions or suspensions, (lipid) emulsions, dispersible powders, suppositories, ointments, creams, drops and sterile injectable aqueous or oily solutions or suspensions.

A suitable pharmaceutical composition of this invention is one suitable for oral administration in unit dosage form, for example a tablet or capsule which contains between 0.1 mg and 1 g of active ingredient.

In another aspect a pharmaceutical composition of the invention is one suitable for intravenous, subcutaneous or intramuscular injection.

Each patient may receive, for example, an intravenous, subcutaneous or intramuscular dose of 0.01 mgkg⁻¹ to 100 mgkg⁻¹ of the compound, preferably in the range of 0.1 mgkg⁻¹ to 20 mgkg⁻¹ of this invention, the composition being administered 1 to 4 times per day. The intravenous, subcutaneous and intramuscular dose may be given by means of a bolus injection. Alternatively the intravenous dose may be given by continuous infusion over a period of time. Alternatively each patient will receive a daily oral dose which is approximately equivalent to the daily parenteral dose, the composition being administered 1 to 4 times per day.

The following illustrate representative pharmaceutical dosage forms containing the compound of formula (I), (Ia), (Ib) or (Ic) {for example (I), (Ia) or (Ib)}, or a pharmaceutically acceptable salt thereof or a solvent thereof (hereafter Compound X), for therapeutic or prophylactic use in humans:

(a)

| Tablet I | mg/tablet |
|---|---|
| Compound X | 100 |
| Lactose Ph. Eur. | 1.79 |
| Croscarmellose sodium | 12.0 |
| Polyvinylpyrrolidone | 6 |
| Magnesium stearate | 3.0 |

(b)

| Tablet II | mg/tablet |
|---|---|
| Compound X | 50 |
| Lactose Ph. Eur. | 229 |
| Croscarmellose sodium | 12.0 |
| Polyvinylpyrrolidone | 6 |
| Magnesium stearate | 3.0 |

(c)

| Tablet III | mg/tablet |
|---|---|
| Compound X | 1.0 |
| Lactose Ph. Eur. | 92 |
| Croscarmellose sodium | 4.0 |
| Polyvinylpyrrolidone | 2.0 |
| Magnesium stearate | 1.0 |

(d)

| Capsule | mg/capsule |
|---|---|
| Compound X | 10 |
| Lactose Ph. Eur. | 389 |
| Croscarmellose sodium | 100 |
| Magnesium stearate | 1.0 |

(e)

| | |
|---|---|
| Injection I | (50 mg/ml) |
| Compound X | 5.0% w/v |
| Isotonic aqueous solution | to 100% |

Buffers, pharmaceutically-acceptable cosolvents such as polyethylene glycol, polypropylene glycol, glycerol or ethanol or complexing agents such as hydroxy-propyl β-cyclodextrin may be used to aid formulation.

The above formulations may be obtained by conventional procedures well known in the pharmaceutical art. The tablets (a)-(c) may be enteric coated by conventional means, for example to provide a coating of cellulose acetate phthalate.

The invention further relates to combination therapies or compositions wherein a compound of formula (I), or a pharmaceutically acceptable salt, solvate or a solvate of a salt thereof, or a pharmaceutical composition comprising a compound of formula (I), or a pharmaceutically acceptable salt, solvate or a solvate of a salt thereof, is administered concurrently (possibly in the same composition) or sequentially with an agent for the treatment of any one of the above disease states.

In particular, for the treatment of the inflammatory diseases rheumatoid arthritis, psoriasis, inflammatory bowel disease, COPD, asthma and allergic rhinitis a compound of the invention can be combined with a TNF-α inhibitor (such as an anti-TNF monoclonal antibodie (such as Remicade, CDP-870 and D.sub2.E.sub7.), or a TNF receptor immunoglobulin molecule (such as Enbrel.reg.)), a non-selective COX-1/COX-2 inhibitor (such as piroxicam or diclofenac; a propionic acid such as naproxen, flubiprofen, fenoprofen, ketoprofen or ibuprofen; a fenamate such as mefenamic acid, indomethacin, sulindac or apazone; a pyrazolone such as phenylbutazone; or a salicylate such as aspirin), a COX-2 inhibitor (such as meloxicam, celecoxib, rofecoxib, valdecoxib or etoricoxib) low dose methotrexate, lefunomide; ciclesonide; hydroxychloroquine, d-penicillamine or auranofin, or parenteral or oral gold.

The present invention still further relates to the combination of a compound of the invention together with:
a leukotriene biosynthesis inhibitor, a 5-lipoxygenase (5-LO) inhibitor or a 5-lipoxygenase activating protein (FLAP) antagonist, such as zileuton, ABT-761, fenleuton, tepoxalin, Abbott-79175, Abbott-85761, an N-(5-substituted)-thiophene-2-alkylsulfonamide, a 2,6-di-tert-butylphenol hydrazones, a methoxytetrahydropyran such as Zeneca ZD-2138, SB-210661, a pyridinyl-substituted 2-cyanonaphthalene compound such as L-739,010; a 2-cyanoquinoline compound such as L-746,530; an indole or quinoline compound such as MK-591, MK-886 or BAY x 1005;
a receptor antagonist for a leukotriene LTB.sub4., LTC.sub4., LTD.sub4. or LTE.sub4. selected from the group consisting of a phenothiazin-3-one such as L-651,392; an amidino compound such as CGS-25019c; a benzoxalamine such as ontazolast; a benzenecarboximidamide such as BIIL 284/260; or a compound such as zafirlukast, ablukast, montelukast, pranlukast, verlukast (MK-679), RG-12525, Ro-245913, iralukast (CGP 45715A) or BAY x 7195;
a PDE4 inhibitor including an inhibitor of the isoform PDE4D;
an antihistaminic H.sub1. receptor antagonist such as cetirizine, loratadine, desloratadine, fexofenadine, astemizole, azelastine or chlorpheniramine;
a gastroprotective H.sub2. receptor antagonist;
an α.sub1.- and α.sub2.-adrenoceptor agonist vasoconstrictor sympathomimetic agent, such as propylhexedrine, phenylephrine, phenylpropanolamine, pseudoephedrine, naphazoline hydrochloride, oxymetazoline hydrochloride, tetrahydrozoline hydrochloride, xylometazoline hydrochloride or ethylnorepinephrine hydrochloride;
an anticholinergic agent such as ipratropium bromide, tiotropium bromide, oxitropium bromide, pirenzepine or telenzepine;
a β.sub1.- to β.sub4.-adrenoceptor agonist such as metaproterenol, isoproterenol, isoprenaline, albuterol, salbutamol, formoterol, salmeterol, terbutaline, orciprenaline, bitolterol mesylate or pirbuterol, or a methylxanthanine including theophylline and aminophylline; sodium cromoglycate; or a muscarinic receptor (M1, M2, and M3) antagonist;
an insulin-like growth factor type I (IGF-1) mimetic;
an inhaled glucocorticoid with reduced systemic side effects, such as prednisone, prednisolone, flunisolide, triamcinolone acetonide, beclomethasone dipropionate, budesonide, fluticasone propionate or mometasone furoate;
an inhibitor of a matrix metalloprotease (MMP), such as a stromelysin, a collagenase, or a gelatinase or aggrecanase; such as collagenase-1 (MMP-1), collagenase-2 (MMP-8), collagenase-3 (MMP-13), stromelysin-1 (MMP-3), stromelysin-2 (IP-10), and stromelysin-3 (MMP-11) or MM-12;
a modulator of chemokine receptor function such as CCR1, CCR2, CCR2A, CCR2B, CCR3, CCR4, CCR5, CCR6, CCR7, CCR8, CCR9, CCR10 and CCR11 (for the C-C family); CXCR1, CXCR2, CXCR3, CXCR4 and CXCR5 (for the C-X-C family) and CX₃CR1 for the C-X₃-C family;
an osteoporosis agent such as roloxifene, droloxifene, lasofoxifene or fosomax;
an immunosuppressant agent such as FK-506, rapamycin, cyclosporine, azathioprine or methotrexate; or,
an existing therapeutic agent for the treatment of osteoarthritis, for example a non-steroidal anti-inflammatory agent (hereinafter NSAID's) such as piroxicam or diclofenac, a propionic acid such as naproxen, flubiprofen, fenoprofen, ketoprofen or ibuprofen, a fenamate such as mefenamic acid, indomethacin, sulindac or apazone, a pyrazolone such as phenylbutazone, a salicylate such as aspirin, a COX-2 inhibitor such as celecoxib, valdecoxib, rofecoxib or etoricoxib, an analgesic or intra-articular therapy such as a corticosteroid or a hyaluronic acid such as hyalgan or synvisc, or a P2X7 receptor antagonist.

The present invention still further relates to the combination of a compound of the invention together with: (i) a tryptase inhibitor; (ii) a platelet activating factor (PAF) antagonist; (iii) an interleukin converting enzyme (ICE) inhibitor; (iv) an IMPDH inhibitor; (v) an adhesion molecule inhibitor including a VLA-4 antagonist; (vi) a cathepsin; (vii) a MAP kinase inhibitor; (viii) a glucose-6 phosphate dehydrogenase inhibitor; (ix) a kinin-B.sub1.- and B.sub2.-receptor antagonist; (x) an anti-gout agent, e.g., colchicine; (xi) a xanthine oxidase inhibitor, e.g., allopurinol; (xii) an uricosuric agent, e.g., probenecid, sulfinpyrazone or benzbromarone; (xiii) a growth hormone secretagogue; (xiv) a transforming growth factor (TGFβ); (xv) a platelet-derived growth factor (PDGF); (xvi) a fibroblast growth factor, e.g., basic fibroblast growth factor (bFGF); (xvii) a granulocyte macrophage colony stimulating factor (GM-CSF); (xviii) a capsaicin cream; (xix) a Tachykinin NK.sub1. and NK.sub3. receptor antagonist selected from the group consisting of NKP-608C; SB-233412 (talnetant); and D4418; (xx) an elastase inhibitors selected from the group consisting of UT-77 and ZD-0892; (xxi) a TNFα converting enzyme inhibitor (TACE); (xxii) an induced nitric oxide synthase inhibitor (iNOS); or (xxiii) a chemoattractant receptor-homologous molecule expressed on TH2 cells (a CRTH2 antagonist).

The invention will now be illustrated by the following non-limiting examples in which, unless stated otherwise:

(i) temperatures are given in degrees Celsius (° C., operations were carried out at room or ambient temperature, that is, at a temperature in the range of 18-25° C.;

(ii) organic solutions were dried over anhydrous magnesium sulphate; evaporation of solvent was carried out using a rotary evaporator under reduced pressure (600-4000 Pascals; 4.5-30 mm Hg) with a bath temperature of up to 60° C.;

(iii) chromatography unless otherwise stated means flash chromatography on silica gel; thin layer chromatography (TLC) was carried out on silica gel plates; where a “Bond Elut” column is referred to, this means a column containing 10 g or 20 g of silica of 40 micron particle size, the silica being contained in a 60 ml disposable syringe and supported by a porous disc, obtained from Varian, Harbor City, Calif., USA under the name “Mega Bond Elut SI”.

(iv) in general, the course of reactions was followed by TLC and reaction times are given for illustration only;

(v) yields, when given, are for illustration only and are not necessarily those which can be obtained by diligent process development; preparations were repeated if more material was required;

(vi) when given, ¹H NMR data is quoted and is in the form of delta values for major diagnostic protons, given in parts per million (ppm) relative to tetramethylsilane (TMS) as an internal standard, determined at 300 MHz using perdeuterio DMSO (CD₃SOCD₃) as the solvent unless otherwise stated; coupling constants (J) are given in Hz;

(vii) chemical symbols have their usual meanings; SI units and symbols are used;

(viii) solvent ratios are given in percentage by volume;

(ix) mass spectra (MS) were run with an electron energy of 70 electron volts in the chemical ionisation (APCI) mode using a direct exposure probe; where indicated ionisation was effected by electrospray (ES); where values for m/z are given, generally only ions which indicate the parent mass are reported, and unless otherwise stated the mass ion quoted is the positive mass ion—(M+M)⁺;

(x) LCMS characterisation was performed using a pair of Gilson 306 pumps with Gilson 233 XL sampler and Waters ZMD4000 mass spectrometer. The LC comprised water symmetry 4.6×50 column C18 with 5 micron particle size. The eluents were: A, water with 0.05% formic acid and B, acetonitrile with 0.05% formic acid. The eluent gradient went from 95% A to 95% B in 6 minutes. Where indicated ionisation was effected by electrospray ES); where values for m/z are given, generally only ions which indicate the parent mass are reported, and unless otherwise stated the mass ion quoted is the positive mass ion—(M+H)⁺ and

(xi) the following abbreviations are used:

DMSO dimethyl sulphoxide;

DMF N-dimethylformamide;

DCM dichloromethane;

HATU O-(7-Azabenzotriazol-1-yl)-N,N′,N′-tetramethyluronium hexafluorophosphate;

HOBT 1-hydroxybenzotriazole hydrate;

EDAC 1-ethyl-3-(3-dimethylaminopropyl)carbodiimide hydrochloride

DIPEA N,N-diisopropylethylamine

EtOH ethanol; and

EtOAc ethyl acetate.

### EXAMPLE 1

This Example illustrates the preparation of (S)-1-acetyl-piperidine-4-carboxylic acid [3-(4-{ethyl-[2-(4-methanesulfonyl-phenyl)-acetyl]-amino}-piperidin-1-yl)-1-phenyl-propyl]-amide (Compound No. 1 of Table I).

To a stirred solution of (S)-N-[1-(3-amino-3-phenyl-propyl)-piperidin-4-yl]-N-ethyl-2-(4-methanesulfonyl-phenyl)-acetamide dihydrochloride (Method B, 0.22 g, 0.42 mmol) in DCM (5 mL) was added N,N-diisopropylethylamine (0.75 mL), 1-acetylpiperidine4-carboxylic acid (100 mg, 0.58 mmol) and HATU (300 mg) and the resulting mixture was stirred at room temperature for 18 h. The mixture was partitioned between water and DCM, the organic phase was washed with water and brine, dried (MgSO₄) and concentrated. The residue was purified by silica column chromatography (eluent 5% MeOH/DCM) to yield the title compound which was characterised by LCMS: 611 (MH+).

### EXAMPLE 2

This Example illustrates the preparation of (S)-4-[3-(4{ethyl-[2-(4-methanesulfonyl-phenyl)-acetyl]-amino}-piperidin-1-yl)-1-phenyl-propylcarbamoyl]-piperidine-1-carboxylic acid tert-butyl ester (Compound No. 2 of Table I).

To a stirred solution of 1-tert-butyloxycarbonylpiperidine-4-carboxylic acid (Method F; 3.5 g, 15 mmol) in DCM (30 mL) was added EDAC (2.87 g, 15 mmol), HOBT (2.03 g, 15 mmol) and triethylamine (2.1 mL, 15 mmol) and the resulting mixture was stirred at room temperature for 5 min. To this mixture was added a solution of (S)-N-[1-(3-amino-3-phenyl-propyl)-piperidin-4-yl]-N-ethyl-2-(4-methanesulfonyl-phenyl)-acetamide dihydrochloride (Method B, 5.3 g, 10 mmol) and triethylamine (2.8 mL, 20 mmol) in DCM (30 mL) and the resulting mixture was stirred at room temperature for 18 h. The mixture was evaporated and the residue partitioned between water and DCM, the organic phase was washed with brine, dried (MgSO₄) and concentrated to give the title compound (5.9 g, 88%).

¹H NMR: 1.0 and 1.1 (t, 3H), 1.4 (s, 9H), 1.5-1.7 (m, 10H), 1.9 (m, 2H), 2.3 (m, 4H), 2.8 (m, 4H), 3.2 (s, 3H), 3.2 and 3.3 (q, 2H), 3.6 and 4.1 (m, 1H), 3.8 and 3.85 (s, 2H), 3.9 (m, 1H), 4.8 (m, 1H), 7.2 (m, 1H), 7.3 (m, 4H), 7.5 (d, 2H), 7.85 (d, 2H), 8.3 (m, 1H).

LCMS: 669 (MH+).

### EXAMPLE 3

This Example illustrates the preparation of (S)-1-benzenesulfonyl-piperidine-4 carboxylic acid [3-(4-{ethyl-[2-(4-methanesulfonyl-phenyl)-acetyl]-amino}-piperidin-1-yl)-1-phenyl-propyl]-amide (Compound No. 3 of Table 1).

To a stirred solution of (S)-piperidine-4-carboxylic acid [3-(4-{ethyl-[2-(4-methanesulfonyl-phenyl)-acetyl]-amino}-piperidin-1-yl)-1-phenyl-propyl]-amide (Method E, 248 mg, 0.45 mmol) and triethylamine (0.14 mL, 1.0 mmol) in DCM (2 mL) under argon was added a solution of benzenesulfonyl chloride (89 mg, 0.5 mmol) in DCM (3 mL) and the resulting mixture was stirred at room temperature for 18 h. The reaction mixture was partitioned between DCM and water and the organic phase was evaporated. The residue was purified by Bond Elut (gradient elution, ethyl acetate to 1:1 methanol/ethyl acetate) giving the title compound (284 mg, 83%).

¹H NMR: 1.0 and 1.1 (t, 3H), 1.5-1.7 (m, 10H), 1.9 (m, 2H), 2.2 (m, 4H), 2.3 (m, 2H), 2.8 (m, 2H), 3.2 (s, 3H), 3.2 and 3.3 (q, 2H), 3.6 and 4.1 (m, 1H), 3.6 (m, 1H), 3.8 and 3.9 (s, 2H), 4.8 (m, 1H), 7.2 (m, 6H), 7.5 (m, 2H), 7.6 (m, 2H), 7.7 (m, 2H), 7.85 (d, 2H), 8.2 (m, 1H).

LCMS: 709 (MH+).

The procedure described in Example 3 can be repeated using different sulfonyl chlorides (such as methanesulfonyl chloride, ethanesulfonyl chloride, 4-methanesulfonylbenzenesulfonyl chloride, 4-fluorobenzenesulfonyl chloride etc.) in place of benzenesulfonyl chloride.

### EXAMPLE 4

This Example illustrates the preparation of (S)-1-isobutyryl-piperidine-4-carboxylic acid [3-(4-{ethyl-[2-(4-methanesulfonyl-phenyl)-acetyl]-amino}-piperidin-1-yl)-1-phenyl-propyl]-amide (Compound No. 11 of Table I).

To a stirred solution of isobutyryl chloride (54 mg, 0.50 mmol) in DCM (1 mL) was added a solution of (S)-piperidine-4-carboxylic acid [3-(4-{ethyl-[2-(4-methanesulfonyl-phenyl)-acetyl]-amino}-piperidin-1-yl)-1-phenyl-propyl]-amide (Method E, 193 mg, 0.34 mmol) and triethylamine (93 μL, 0.68 mmol) in DCM (2 mL) and the resulting mixture was stirred at room temperature for 18 h. The mixture was partitioned between DCM and 2N sodium hydroxide solution and the organic phase was evaporated. The residue was purified by Bond Elut (gradient elution, ethyl acetate to 1:1 methanol/ethyl acetate) giving the title compound (142 mg).

¹H NMR: 0.95 (d, 6H), 1.0 and 1.1 (t, 3H), 1.25-1.55 (m, 4H), 1.62.0 (m, 8H), 2.2 (m, 2H), 2.45 (m, 2H), 2.8 (m, 2H), 3.0 (m, 1H), 3.2 (s, 3H), 3.2 and 3.35 (q, 2H), 3.3 (m, 1H), 3.6 and 4.1 (m, 1H), 3.8 and 3.9 (s, 2H), 3.95 (m, 1H), 4.4 (m, 1H), 4.8 (m, 1H), 7.2 (m, 1H), 7.3 (m, 4H), 7.5 (m, 2H), 7.85 (d, 2H), 8.3 (m, 1H).

LCMS: 639 (MH+).

The procedure described in Example 4 can be repeated using different acid chlorides (such as phenylacetyl chloride, benzoyl choride, cyclopropanecarbonyl chloride, 4-chlorobenzoyl chloride etc.) in place of isobutyryl chloride.

### EXAMPLE 5

This Example illustrates the preparation of (S)-[3-(4-{ethyl-[2-(4-methanesulfonyl-phenyl)-acetyl]-amino}-piperidin-1-yl)-1-phenyl-propyl]-carbamic acid tert-butyl ester (Compound No. 1 of Table II).

To a stirred solution of (S)-(3-oxo-1-phenyl-propyl)-carbamic acid tert-butyl ester (Method C, 25 g, 100 mmol), N-ethyl-2-(4-methanesulfonyl-phenyl)-N-piperidin-4-yl-acetamide (Method A, 36 g, 110 mmol) and glacial acetic acid (6.0 mL) in a 1:1 mixture of DCM and methanol (500 mL) was added portionwise sodium triacetoxyborohydride (25 g, 120 mmol) at ambient temperature. The reaction mixture was stirred for a further 12 hours, then 2N sodium hydroxide solution (500 mL) was cautiously added during 30 minutes and the resulting mixture extracted into DCM (500 mL). The crude product was purified by chromatography on silica eluting with a 9:1 mixture of ethyl acetate and methanol to give the title compound as a colourless gum, which slowly solidified (40 g). A portion was crystallised from ethyl acetate to give a white solid, m.p. 115-116° C.

¹H NMR: 1.0 and 1.1 (t, 3H), 1.35 (s, 9H), 1.5 (m, 2H), 1.7 (m, 4H), 1.9 (m, 2H), 2.2 (t, 2H), 2.8 (m, 2H), 3.15 (s, 3H), 3.2 and 3.3 (q, 2H), 3.6 and 4.1 (m, 1H), 3.8 and 3.85 (s, 2H), 4.5 (m, 1H), 7.2 (m, 5H), 7.4 (br d, 1H), 7.5 (d, 2H), 7.8 (d, 21[).

LCMS: 558 (MH+).

### EXAMPLE 6

This Example illustrates the preparation of (S)-[3-(4-{ethyl-[2-(4-methanesulfonyl-phenyl)-acetyl]-amino}-piperidin-1-yl)-1-phenyl-propyl]-carbamic acid 1′,1′,1′-trichloroethoxy ester hydrochloride (Compound No. 7 of Table II).

To a stirred solution of (S)-N-[1-(3-Amino-3-phenyl-propyl)-piperidin-4-yl]-N-ethyl-2-(4-methanesulfonyl-phenyl)-acetamide dihydrochloride (Method B, 0.16 g, 3 mmol) in DCM (10 mL) was added triethylamine (0.13 mL, 9 mmol) and the reaction was cooled to 0° C. under an atmosphere of argon. Trichloroethyl chloroformate (65 mg, 0.3 mmol) was then added in one portion, and the reaction mixtures were stirred overnight. The mixture was then filtered through a diatomaceous earth cartridge, which had been preloaded with saturated sodium bicarbonate solution, followed by a second cartridge loaded with 1N HCl. The crude product was purified by Bond Elut (dichloromethane then 5% methanol in DCM) and the resulting product was isolated as an HCl salt by addition of 1N HCl in diethyl ether followed by tituration to give the title compound as a white solid (130 mg, 65%).

¹H NMR: 1.3 (br s, 3H), 1.9 (br, 2H), 2.4 (br m, 1H), 2.6 (br m, 3H), 2.8 (br m, 2H), 3.0 (br m, 1H), 3.1 (m, 4H), 3.4 (br m, 2H), 3.6 (br m, 2H), 3.8 (s, 2H), 4.7 (m, 4H), 6.2 (m, 1H), 7.3 (m, 5H), 7.4 (d, 2H), 7.9 (d, 2H), 12.5 (br s, 1H).

LCMS: 634 (MH+).

The procedure described in Example 6 can be repeated using different chloroformates (such as phenyl chloroformate, benzyl chloroformate, methoxyethyl chloroformate, 4-fluorophenyl chloroformate etc.) in place of trichloroethyl chloroformate.

### EXAMPLE 7

This Example illustrates the preparation of (S)-[3-(4-{ethyl-[2-(4-methanesulfonyl-phenyl)-acetyl]-amino}-piperidin-1-yl)-1-(3-fluoro-phenyl)-propyl]-carbamic acid tert-butyl ester (Compound No. 11 of Table II).

To a solution of (S)-[1-(3-fluoro-phenyl)-3-oxo-propyl]-carbamic acid tert-butyl ester (Method D, 0.85 g, 3.12 mmol) in DCM (70 mL) and N-ethyl-2-(4-methanesulfonyl-phenyl)-N-piperidin-4-yl-acetamide (Method A, 1.19 g, 3.67 mmol) was added glacial acetic acid (one drop) and the resulting mixture was stirred at room temperature for 1 h. Sodium triacetoxyborohydride (1.4 g, 6.4 mmol) was added and the resulting mixture was stirred at room temperature for 18 h. The reaction mixture was quenched with water and the organic phase was washed with sodium hydrogen carbonated solution (saturated aqueous) and water, dried (MgSO₄) and concentrated. The crude product was purified by Bond Elut (ethyl acetate then 8% methanol in ethyl acetate) to give the title compound as a solid (1.00 g, 55%).

¹H NMR: 1.0 and 1.1 (t, 3H), 1.35 (s, 9H), 1.5 (m, 2H), 1.7 (m, 4H), 1.9 (m, 2H), 2.2 (t, 2H), 2.8 (m, 2H), 3.2 (s, 3H), 3.2 and 3.3 (q, 2H), 3.6 and 4.1 (m, 1H), 3.8 and 3.85 (s, 2H), 4.5 (m, 1H), 7.05 (m, 1H), 7.1 (m, 2H), 7.35 (dd, 1H), 7.5 (br d, 1H), 7.5 (d, 2H), 7.85 (d, 2H).

LCMS: 576 (MH+).

Starting materials are commercially available, have been described in the literature or can be prepared by adaptation of literature methods. Examples of literature methods include: P. Richter, Ch. Garbe and G. Wagner, E. Ger. Pharmazie, 1974, 29(4), 256262; C. Oniscu, D. Nicoara and G. Funieru, “4-(Ureidosulfonyl)phenylacetic acid and its ureide.”, RO79-966646, (Romanian document); and M. A. Zahran, M. M. Ali, Y. A. Mohammed and A. A Shehata, Int. J. Chem., 1993, 4(3), 61.

Method A

### N-Ethyl-2-(4-methanesulfonyl-phenyl)-N-piperidin-4-yl-acetamide

### Step 1: Preparation of (1-benzyl-piperidin-4-yl)-ethyl-amine dihydrochloride

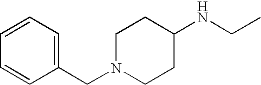

To a solution of 1-phenylmethyl-4-piperidone (25.0 g, 132 mmol) in THF (250 mL) was added ethylamine hydrochloride (12.0 g, 147 mmol) and methanol (50 mL) and the resulting mixture stirred at room temperature for 10 min. Sodium triacetoxyborohydride (40 g, 189 mmol) was added portionwise and the resulting mixture stirred at room temperature for 1 h. 2M Sodium hydroxide solution (250 mL) was added and the resulting mixture extracted with diethyl ether. The organic extracts were dried (K₂CO₃) and evaporated to give 1-phenylmethyl-4-ethylaminopiperidine as an oil. This was dissolved in ethanol (500 mL) and concentrated hydrochloric acid (20 mL) was added. The resulting crystals were collected, washed with diethyl ether and dried giving the sub-titled compound as a solid (38 g).

¹H NMR: (CDCl₃): 1.10 (t, 3H), 1.40 (m, 2H), 1.83 (m, 2H), 2.02 (m, 2H), 2.65 (q, 2H), 2.85 (m, 2H), 3.50 (s, 2H), 3.75 (m, 1H), 7.2-7.4 (m, 5H).

MS: 219

### Step 2: Preparation of N-(1-benzyl-piperidinyl)-N-ethyl-2-(4-methanesulfonyl-phenyl)-acetamide

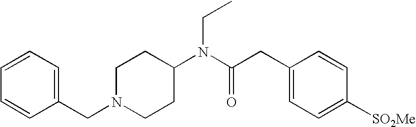

To a solution of (1-benzyl-piperidinyl)-ethyl-amine dihydrochloride (32.0 g, 110 mmol) in DCM (500 mL) was added N,N-diisopropylethylamine (60 mL) with stirring to ensure complete dissolution. 4-Methanesulfonylphenylacetic acid (25.0 g, 117 mmol), 4-Dimethylaminopyridine (4-DMAP) (2.0 g) and dicyclohexylcarbodiimide (DCCI) (25.0 g, 121 mmol) were added and the resulting mixture was stirred at room temperature for 20 h. The precipitate was removed by filtration and the resulting solution was washed successively with 2N aqueous HCl, water and 1N aqueous NaOH, dried (MgSO₄) and evaporated. The residue was purified by silica gel chromatography (eluent 10% MeOH/ethyl acetate) to afford the sub-titled compound (35 g, 76%).

¹H NMR: 1.00 and 1.14 (t, 3H), 1.45 and 1.70 (m, 2H), 1.95 (br m, 2H), 2.80 (br m, 2H), 3.18 (s, 3H), 3.20 and 3.33 (q, 2H), 3.5 (s, 2H), 3.80 and 3.87 (s, 2H), 3.70 and 4.10 (m, 1H), 7.2-7.3 (m, 5H), 7.48 (m, 2H), 7.82 (m, 2H).

MS: 415 (MH+).

### Step 3: Preparation of Title Compound

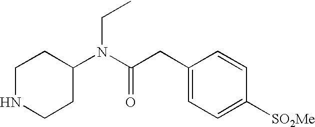

To a solution of N-(1-benzyl-piperidinyl)-N-ethyl-2-(4-methanesulfonyl-phenyl)-acetamide (34 g, 82 mmol) in ethanol (600 mL) was added ammonium formate (40 g). The mixture was purged with argon and 30% Pd on carbon (4.2 g) was added. The resulting mixture was stirred at reflux for 4 h, then allowed to cool and filtered through diatomaceous earth. The filtrate was evaporated to give a thick oil which solidified on standing to yield the titled compound (24.9 g, 94%).

¹H NMR: 1.02 and 1.15 (t, 3H), 1.4-1.6 (br m, 4H), 2.45 (m, 2H), 2.93 (br m, 2H), 3.18 (s, 3H), 3.20 and 3.32 (q, 2H), 3.72 and 4.18 (m, 1H), 3.80 and 3.87 (s, 2H), 7.50 (m, 2H), 7.85 (m, 2H).

MS: 325 (MH+).

Method B

### Preparation of (S)-N-(1-(3-Amino-3-phenyl-propyl)-piperidin-4-yl]-N-ethyl-2-(4-methanesulfonyl-phenyl)-acetamide dihydrochloride

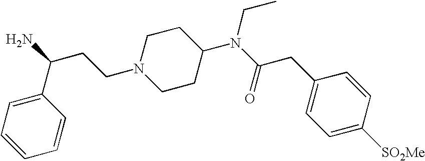

(S)-[3-(4-{ethyl-[2-(4-methanesulfonyl-phenyl)-acetyl]-amino)-piperidin-1-yl)-1-phenyl-propyl]-carbamic acid tert-butyl ester (Example 5, 20 g) was suspended in DCM (200 mL) and a solution of hydrochloric acid in methanol (2M, 50 mL) was added. The resulting mixture was stirred for 1 h then evaporated yielding the title compound as a white solid (19 g).

¹H NMR (d6 DMSO at 373K): 1.1 (t, 3H), 1.5 (m, 2H), 1.9 (m, 2H), 2.0 (m, 1H), 2.3 (m, 2H), 3.0 (m, 1H), 3.2 (m, 4H), 3.3 (q, 2H), 3.9 (s, 2H), 4.0 (m, 1H), 4.4 (m, 1H), 7.4 (m, 3H), 7.5 (m, 4H), 7.9 (m, 2H).

MS: 458.

Method C

### (S)-(3-Oxo-1-phenyl-propyl)-carbamic acid tert-butyl ester

### Step 1: Preparation of (S)-N-methyl-N-methoxy-3-phenyl-3-Bocaminopropionamide

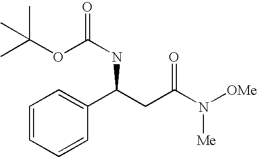

To a solution of (S)-3-phenyl-3-Bocaminopropanoic acid (available from PepTech Corp. of Cambridge, Mass., USA; 4.97 g, 18.7 mmol) in DCM (100 mL) was added DIPEA (14.8 mL, 84.8 mmol) and N,O-dimethylhydroxylamine hydrochloride (2.21 g, 22.7 mmol) followed by HATU (8.44 g, 84.8 mmol). The resulting mixture was stirred at room temperature for 18 h, diluted with DCM, washed with 2M aqueous sodium hydroxide and water. The organic phase was dried (Na₂SO₄) and concentrated. The residue was purified by silica column chromatography (eluting with isohexane then 3:1 ethyl acetate to isohexane) giving the sub-title compound as a colourless oil (5.58 g, 97%).

¹H NMR (CDCl₃): 1.40 (s, 9H), 2.83 (dd, 1H), 3.01 (m, 1H), 3.08 (s, 3H), 3.52 (s, 3H), 5.10 (m, 1H), 7.28 (m, 5H).

MS: 309.

### Step 2: Preparation of Title Compound

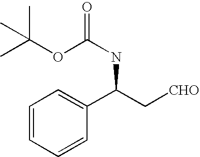

To a solution of (S)-N-methyl-N-methoxy-3-phenyl-3-Bocaminopropionamide (5.52 g, 17.9 mmol) in toluene (180 mL) at −20° C. was added sodium bis(2-methoxyethoxy)aluminium hydride (65% solution in toluene, 35.8 mmol) dropwise. The resulting mixture was stirred at −15° C. for 1 h. The mixture was washed with saturated aqueous sodium dihydrogen phosphate solution (250 mL). The organic phase was dried (Na₂SO₄) and concentrated to give the title compound (5 g).

¹H NMR: 1.4 (s, 9H), 2.8 (m, 2H), 5.1 (m, 1H), 7.3 (m, 5H), 8.6 (m, 1H), 9.6 (t, 1H).

Method D

### (S)-[1-(3-Fluoro-phenyl)-3-oxo-propyl]-carbamic acid tert-butyl ester

### Step 1: Preparation of trans-3-fluorocinnamic acid tert-butyl ester

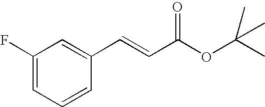

To a stirred solution of trans-3-fluorocinnamic acid (4.34 g, 26.1 mmol) in toluene (40 mL) at 110° C. was added N,N-dimethylformamide di-tert-butyl acetal (25 mL, 104 mmol) dropwise over 30 min. The resulting mixture was stirred at reflux for a further 4 h. The mixture was then cooled to room temperature and washed with water (50 mL), saturated aqueous sodium hydrogen carbonate solution (2×100 mL) and brine (10 mL), dried (MgSO₄) and evaporated. The crude product was purified by Bond Elut (isohexane then 2% ethyl acetate in isohexane) to give the sub-title compound as a liquid (3.7 g, 64%).

### Step 2: Preparation of (3S,1′R)-3-[benzyl-(1-phenyl-ethyl)-amino]-3-(3-fluoro-phenyl)-propionic acid tert-butyl ester

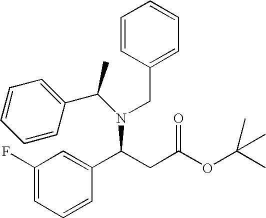

To a stirred solution of (R)-(+)-N-benzyl-α-methylbenzylamine (4.0 mL, 19 mmol) in THF (20 mL) at −78° C. was added n-butyl lithium (1.6M in hexanes, 12.5 mL, 20 mmol) and the resulting mixture was allowed to warm to room temperature over 10 min. before recooling to −78° C.; A solution of trans-3-fluorocinnamic acid tert-butyl ester (3.74 g, 16.8 mmol) in THF (200 mL) was added and the resulting mixture was stirred at −78° C. for 2 h then quenched by the addition of saturated aqueous ammonium chloride solution (25 mL). After warming to room temperature the organic phase was washed with water (2×50 mL) and brine, dried (MgSO₄) and evaporated. The crude product was purified by Bond Elut (isohexane then 2% ethyl acetate in isohexane) to give the sub-title compound as a gum (5.85 g, 80%).

¹H NMR (400 MHz, CDCl₃): 1.23 (s, 9H), 1.27 (d, 3H), 2.48 (m, 2H), 3.67 (s, 2H), 3.97 (q, 1H), 4.40 (dd, 1H), 6.93 (ddd, 1H), 7.1-7.4 (m, 13H).

### Step 3: Preparation of 3-tert-butoxycarbonylamino-3-(3-fluoro-phenyl)-propionic acid tert-butyl ester

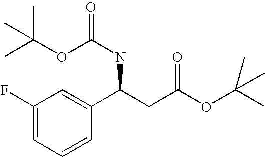

A stirred mixture of (3S,1′R)-3-[benzyl-(1-phenyl-ethyl)-amino]-3-(3-fluoro-phenyl)-propionic acid tert-butyl ester (5.39 g, 12.4 mmol), di-tert-butyl dicarbonate (2.98 g, 13.7 mmol) and 20% palladium hydroxide on carbon (0.59 g) in ethanol (100 mL) was hydrogenated at 5 Bar at room temperature for 24 h. The catalyst was removed by filtration through a pad of Celite® washing through with ethanol. The filtrate was evaporated to give an oil which was parititioned between ethyl acetate and saturated aqueous sodium hydrogen carbonate solution. The organic phase was dried (MgSO₄) and evaporated. The crude product was purified by Bond Elut (eluting with isohexane then 5% ethyl acetate in isohexane) to give the sub-title compound as an oil (3.63 g, 86%).

¹H NMR: 1.33 (s, 18H), 2.63 (m, 2H), 4.90 (m, 1H), 7.06 (ddd, 1H), 7.24 (m, 2H), 7.37 (dd, 1H), 7.50 (br d, 1H).

### Step 4: Preparation of (S)-[1-(3-fluoro-phenyl)-3-hydroxy-propyl]-carbamic acid tert-butyl ester

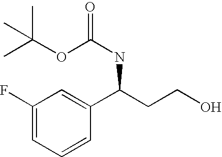

To a stirred, ice-cooled solution of 3-tert-butoxycarbonylamino-3-(3-fluoro-phenyl)-propionic acid tert-butyl ester (2.46 g, 7.25 mmol) in THF (35 mL) was added lithium aluminium hydride (1M in THF, 7.50 mL, 7.50 mmol) dropwise over 20 min. The resulting mixture was stirred with warming to room temperature for 2 h. The reaction was quenched with water (0.275 mL) then 15% aqueous sodium hydroxide (0.275 mL) and more water (0.825 mL) were added with stirring. The resultant precipitate was removed by filtration washing with THF, and the filtrate was dried (MgSO₄) and evaporated. The crude product was purified by Bond Elut (gradient elution, isohexane to 30% ethyl acetate in isohexane) to give the sub-title compound as an oil (1.26 g, 65%).

¹H NMR: 1.4 (s, 9H), 1.75 (m, 1H), 1.85 (m, 1H), 3.3 (m, 1H), 3.4 (m, 1H), 4.5 (dd, 1H), 4.65 (br m, 1H), 7.1 (m+br s, 3H), 7.35 (m, 2H).

### Step 5: Preparation of Title Compound

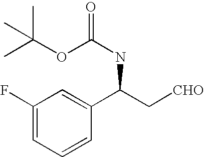

To a solution of (S)-[1-(3-fluoro-phenyl)-3-hydroxy-propyl]-carbamic acid tert-butyl ester (0.85 g, 3.2 mmol) in DCM (70 mL) under argon was added Dess-Martin periodinane (1.48 g, 3.5 mmol) and the resulting mixture was stirred at room temperature for 2 h before the addition of 2M aqueous sodium hydroxide (50 mL). The organic layer was dried (MgSO₄) and evaporated to give the title compound (quantitative).

¹H NMR: 1.4 (s, 9H), 2.8 (m, 2H), 5.1 (m, 1H), 7.05 (ddd, 1H), 7.15 (m, 2H), 7.35 (m, 1H), 7.5 (br d, 1H), 9.6 (s, 1H).

Method E

### (S)-Piperidine-4-carboxylic acid [3-(4 (ethyl-[2-(4-methanesulfonyl-phenyl)-acetyl]-amino}-piperidin-1-yl)-1-phenyl-propyl]-amide

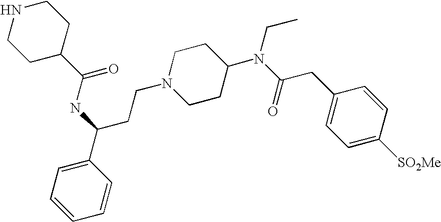

To a stirred solution of (S)-4-[3-(4 (ethyl-[2-(4-methanesulfonyl-phenyl)-acetyl]-amino)-piperidin-1-yl)-1-phenyl-propylcarbamoyl]-piperidine-1-carboxylic acid tert-butyl ester (Example 2, 5.85 g, 8.73 mmol) in DCM (45 mL) was added trifluoroacetic acid (15 mL) and the resulting mixture was stirred at room temperature for 2 h. The mixture was evaporated and the residue partitioned between DCM and 2N sodium hydroxide solution. The organic phase was washed with brine, dried (MgSO₄) and evaporated giving the title compound (4.0 g, 81%).

¹H NMR: 1.0 and 1.1 (t, 3H), 1.5-1.9 (m, 14H), 2.4 (m, 2H), 2.8 (m, 2H), 3.0 (2H), 3.2 (s, 3H), 3.2 and 3.4 (q, 2H), 3.6 and 4.1 (m, 1H), 3.8 and 3.9 (s, 2H), 4.1 (m, 1H), 4.8 (m, 1H), 7.2 (m, 1H), 7.3 (m, 4H), 7.5 (d, 2H), 7.85 (d, 2H), 8.3 (m, 1H).

LCMS: 569 (MH+).

Method F

### 1-tert-Butyloxycarbonylpiperidine-carboxylic acid

To a stirred solution of isonipecotic acid (4.54 g, 35 mmol) in 2N aqueous sodium hydroxide (38.5 mL, 77 mmol) was added portionwise a solution of di-tert-butyl dicarbonate (8.4 g, 38.5 mmol) in THF (20 mL). The resulting mixture was stirred at room temperature for 3 h. The THF was removed by evaporation under reduced pressure and the remaining aqueous mixture was washed with DCM then acidified to pH 4-5 with 1N hydrochloric acid. The resultant precipitate was collected by filtration, washed with water and dried under vacuum at 50° C. affording the title compound.

¹H NMR: 1.35 (m, 2H), 1.4 (s, 9H), 1.8 (m, 2H), 2.4 (m, 1H), 2.8 (m, 2H), 3.8 (m, 2H), 12.2 (br s, 1H).

### EXAMPLE 8

The ability of compounds to inhibit the binding of RANTES or MIP-1α was assessed by an in vitro radioligand binding assay. Membranes were prepared from Chinese hamster ovary cells which expressed the recombinant human CCR5 receptor. These membranes were incubated with 0.1 nM iodinated RANTES or MP-1α, scintillation proximity beads and various concentrations of the compounds of the invention in 96-well plates. The amount of iodinated RANTES or MIP-1α bound to the receptor was determined by scintillation counting. Competition curves were obtained for compounds and the concentration of compound which displaced 50% of bound iodinated RANTES or MIP-1α was calculated (IC₅₀). The compounds of formula (I) had an IC₅₀ of less than 50 μM. For example: Compound 1 of Table I has an IC₅₀ of 39 nM (that is 39 nanoM); Compound 5 of Table I has an IC₅₀ of 28 nM; and, Compound 3 of Table II has an IC₅₀ of 110 nM.

1. A compound of formula (I): 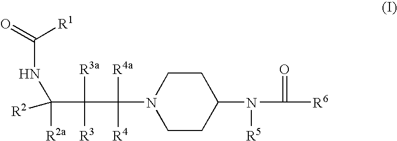
wherein:
R¹ is C₁₋₆ alkoxy; optionally substituted by C₁₋₄ alkoxy or phenyl; C₃₋₆ alkenyloxy, phenoxy or piperidin-4-yl; 1-substituted by C(O)R⁷ or S(O)₂R⁸;
R² is optionally substituted phenyl, optionally substituted heteroaryl or cycloalkyl;
R^{2a}, R⁴ and R^{4a} are, independently, hydrogen or C₁₋₄ alkyl;
R³ and R^{3a} are, independently, hydrogen or C₁₋₄ alkyl or C₁₋₄ alkoxy;
R⁵ is hydrogen, C₁₋₄ alkyl optionally substituted by halogen, hydroxy, C₁₋₄ alkoxy, C₃₋₇ cycloalkyl, SH, C₁₋₄ alkylthio, cyano or S(O)_{q}(C₁₋₄ alkyl); C₃₋₄ alkenyl, C₃₋₄ alkynyl or C₃₋₇ cycloalkyl;
R⁶ is phenyl, heteroaryl, phenylNH, heteroarylNH, phenyl(C₁₋₂)alkyl, heteroaryl(C₁₋₂)alkyl, phenyl(C₁₋₂ alkyl)NH or heteroaryl(C₁₋₂ alkyl)NH;
R⁷ is C₁₋₆ alkyl; optionally substituted by phenyl, heteroaryl, C₁₋₄ alkoxy, or C₁₋₄ alkoxy(C₁₋₄ alkoxy); C₁₋₆ alkoxy, phenyl, heteroaryl or C₃₋₆ cycloalkyl;
R⁸ is C₁₋₆ alkyl; optionally substituted by phenyl; or phenyl;
wherein the phenyl and heteroaryl rings of any of the foregoing are independently optionally substituted by halo, cyano, nitro, hydroxy, C₁₋₄ alkyl, C₁₋₄ alkoxy, S(O)ₘC₁₋₄ alkyl, S(O)₂NR⁹R¹⁰, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃;
R⁹ and R¹⁰ are, independently, hydrogen or C₁₋₄ alkyl, or together with a nitrogen or oxygen atom, may join to form a 5- or 6-membered ring which is optionally substituted with C₁₋₄ alkyl, C(O)H or C(O)(C₁₋₄ alkyl);
m, p and q are, independently, 0, 1 or 2;
or a pharmaceutically acceptable salt thereof or a solvate thereof.

2. A compound as claimed in claim 1 wherein R¹ is piperidin-4-yl 1-substituted by C(O)R⁷; wherein R⁷ is C₁₋₆ alkyl; optionally mono-substituted by phenyl; C₁₋₆ alkoxy, phenyl or C₃₋₆ cycloalkyl, wherein the phenyl rings are optionally substituted by halogen; or S(O)₂R⁸; wherein R⁸ is phenyl or C₁₋₆ alkyl; optionally mono-substituted by phenyl; wherein the phenyl rings are optionally substituted by halogen, S(O)₂(C₁₋₄ alkyl) or NHC(O)(C₁₋₄ alkyl); or R¹ is C₁₋₆ alkoxy; optionally substituted by C₁₋₄ alkoxy or phenyl; C₃₋₆ alkenyloxy or phenoxy; optionally substituted by halogen.

3. A compound as claimed in claim 1 wherein R² is phenyl optionally substituted by halo, C₁₋₄ alkyl, C₁₋₄ alkoxy, S(O)ₙ(C₁₋₄ alkyl); wherein n is 0, 1 or 2; nitro, cyano or CF₃.

4. A compound as claimed in claim 1 wherein R^{2a}, R³, R^{3a}, R⁴ and R^{4a} are all hydrogen.

5. A compound as claimed in claim 1 wherein R⁵ is ethyl.

6. A compound as claimed in claim 1 wherein R⁶ is benzyl singly substituted by S(O)₂(C₁₋₄)alkyl or S(O)₂NR⁹R¹⁰; wherein R⁹ and R¹⁰ are, independently, hydrogen or C₁₋₄ alkyl, or together with a nitrogen or oxygen atom, may join to form a 5- or 6-membered ring which is optionally substituted with C₁₋₄ alkyl, C(O)H or C(O)(C₁₋₄ alkyl).

7. A process for preparing a compound of claim 1, formula (I) comprising:
a) treating a compound of formula (II): 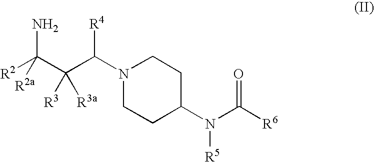
with:
i. an acid chloride or chloroformate of formula R¹C(O)Cl, in the presence of a base and in a suitable solvent; or
ii. when R¹ is a 1-substituted piperidin-4-yl, an acid of formula R¹CO₂H in the presence of a suitable coupling agent in a suitable solvent; OR
b) reacting a compound of formula (VII): 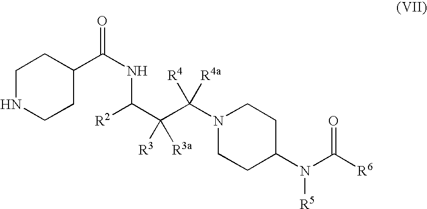
i. with an acid chloride R⁷C(O)Cl or sulfonyl chloride R⁸S(O)₂Cl in the presence of a base and in a suitable solvent; or
ii. with an acid of formula R⁷CO₂H in the presence of a suitable coupling agent in the presence of a suitable base in a suitable solvent.

8. A pharmaceutical composition which comprises a compound of the formula (I), or a pharmaceutically acceptable salt thereof or solvate thereof as claimed in claim 1, and a pharmaceutically acceptable adjuvant, diluent or carrier.

9-10. (canceled)

11. A method of treating a chemokine mediated disease state, comprising administering a compound of formula (I), or a pharmaceutically acceptable salt thereof or solvate thereof as claimed in claim 1.
